(19) United States
(12) Patent Application Publication
(10) Pub. No.: US 2016/0279063 A1
(43) Pub. Date: Sep. 29, 2016

(54) COMPOSITION OF EXCIPIENTS AND PHARMACEUTICAL FORMS WITH SUSTAINED RELEASE AND INCREASED BIOAVAILABILITY OF ANTIBACTERIAL DRUGS, ANTICOCCIDIAL DRUGS AND OTHER DRUGS FOR COMMERCIAL POULTRY AND PIGS

(71) Applicant: UNIVERSIDAD NACIONAL AUTÓNOMA DE MÉXICO, Ciudad Universitaria S/NDistrito Federal (MX)

(72) Inventors: Hector Salvador SUMANO LOPEZ, Colonia San Pablo Tepetlapa (MX); Lilia GUTIERREZ OLVERA, Colonia San Pablo Tepetlapa (MX)

(21) Appl. No.: 14/442,610

(22) PCT Filed: Nov. 14, 2013
(86) PCT No.: PCT/MX2013/000137

§ 371 Date: May 13, 2015

(30) Foreign Application Priority Data
Nov. 14, 2012 (MX) MX/A/2012/013222

(51) Int. Cl. A61K 9/16; A61K 31/665; A61K 31/7048; A61K 31/65; A61K 31/165; A61K 31/50; A61K 31/505; A61K 31/496; A61K 31/221

(52) U.S. Cl. CPC A61K 9/1652; A61K 31/496; A61K 31/665; A61K 31/7048; A61K 31/221; A61K 31/165; A61K 31/50; A61K 31/505; A61K 31/65; A61K 9/1682

(57) ABSTRACT

The invention relates to a composition of excipients and pharmaceutical forms of sustained release and increased drug bioavailability for poultry and pigs and to a method of producing the same, said composition comprises: pharmaceutically active agents, bioavailability promoting agents, polymers for prolonged release of the drug, colouring agents, and flavouring agents. The composition of excipients and pharmaceutical forms of the invention optimizes the dosing of the drug dosage and generates resistant strains of bacteria by optimizing the ratio between pharmacokinetics/pharmacodynamics of drugs. The composition has different forms and colours that allow the product to be identified and accepted more easily by the bird or pig.

### FIELD OF INVENTION

The present invention belongs to veterinary field and refers to development of drug sustained-release pharmaceutical excipients and forms, and more particularly is related to a composition which increases bioavailability and long-action sustained release (FOLA) of antibacterial drugs, analgesics, mucolytics, anticoccidial drugs, vitamins, minerals and other drugs in commercial poultry and pigs.

### BACKGROUND OF INVENTION

In spite of poultry farming high technology level, the use of antimicrobials in the main countries in our continent is far from being the best suitable, and several mistakes in pharmaceutical design of antibiotics, anticoccidial drugs and other drugs for application in poultry have been documented. That above is in fact a significant problem for public health when considering that an unsuitable use of antimicrobials in birds is a potential cause of bacterial resistance leading to a clinical response decrease both in animals and humans by pathogens such as Escherichia coli, Salmonella sp and Campylobacter sp. (Pigan & Kolter, 2002, Lenski, 1998).

Serum profiles of an antimicrobial or antimicrobial activity profile of a drug and its metabolites during a certain time define the way in which an antimicrobial optimally acts in what is called pharmacokinetics/pharmacodynamics ratio or rationality (PK/PD) in antibacterial activity. For example: there is little or null rationality between “antibacterial destination in the organism” and its action mechanism (PK/PD ratio) when polymyxin B or E or neomycin are orally administered for a respiratory condition if they are NOT absorbed. On the other hand, injection of an antibacterial requiring a long permanence in organism (treatments of at least 5 days and where 60% of dosage interval is over CMI every day) to have an optimal antibacterial effect is questionable. Ceftiofur is an example of this suboptimal use which is added to Marek vaccine and is only applied once when an optimal use would be at least 3 days.

Gentamicin is perhaps the antibacterial with faster destruction of bacteria, but in order to note this effect, the rationale should not be in terms of the time that plasma concentration is above of minimum inhibiting concentration (CMI) but instead in terms of achieving 8 to 10 times the CMI value to optimal bactericidal concentration (COB), unfortunately it is not orally absorbed and it has to be necessarily injected, which is impractical in birds and pigs. On the contrary, there are antibacterial drugs which once that they stop bacterial growth with concentrations equivalent to CMI value or from 2 to 4 times CMI value, they do not achieve a faster effect when concentration is increased, in addition to not to achieve it feasibly at reasonable doses in the organism. It is important in these cases that a sufficient concentration is “always” achieved so as to inhibit growth. Thus, the effects of antibacterial concentrations in terms of CMI and COB (optimal bactericidal concentration) provide a description of the antibacterial activity-time and antibacterial activity-concentration relationship of a given antibiotic family, respectively. Depending on these profiles and their clinical efficacy, two antimicrobial action models have been described, those which are concentration-dependent (CD) and those described as with higher efficacy related with its permanence in the organism or time-dependent (TD).

### Time-Dependent Antibacterial Drugs.

The clinical effect is achieved at its optimal expression when the medicament is administered in such way and through a suitable route as to achieve an almost continuous contact between bacteria and antibacterial. Time-dependent (TD) antibacterial drugs are considered β-lactams, macrolides, tetracyclines, sulfonamides, phenicols, phosphomycin, lincomycin and clindamycin. Optimal destruction rate occurs at certain serum and tissue concentration equivalent or preferably above CMI value but during a maximum time between dosage intervals (ID) (T≧CMI at least 75% ID and preferably during the whole ID). Then, it is not better to provide large doses but separate them in several intakes or instead, having a medicament which achieves a sustained release since at larger concentrations (>Cmax) microorganisms are not destroyed neither faster nor extensively. For β-lactams, for example, clinical efficacy is directly related to T≧CMI, when time above or equivalent to CMI is higher than 40 to 50% of dosage interval it may reach up to 60% of clinical and bacteriological efficacy and when T>CMI is from 60 to 70% of dosage interval, it may provide from 80 to 90% of bacteriological and clinical efficacy. If T>CMI is 100 in ID, then a preparation expressing the highest antibacterial potential has been achieved.

Above discussion is applicable to several medicaments in addition to antimicrobials. Clinical efficacy is not added by achieving high concentrations for any drug or vitamin or mixture of microelements if they are DT; what is required is a constant supply for optimal effect and for not to cause toxicity.

### Concentration-Dependent Antibacterial Drugs (CD)

There are antibacterial drugs which efficacy depends more directly on the drug reached concentration in the site of action. Values which shall be reached in plasma and tissues shall be the maximum possible and therefore, bolus doses shall be always delivered (all dose in the least possible time), which is actually a problem in case of birds and pigs. Moreover, high-bioavailability proven quality preparations must be used. It is clearly identified that at higher antibacterial concentration, there is a higher bacterial destruction rate and lower mutant selection. The result is that clinical efficacy will be clearly and rapidly manifested. Thus, the pharmacokinetic variables would be Cmax/CMI of at least: 8 to 10 times for aminoglycosides and >10-12 for fluoroquinolones in case of Gram-positive bacteria and of >10 times for aminoglycosides and >12 for fluoroquinolones for Gram-negative bacteria.

It is also known that they depend on the area under curve rate (AUC) (bioavailability measure) over microorganism CMI being in treatment. This is especially relevant for drugs with long half-life such as fluoroquinolones. Thus, AUC/CMI ratio will be >30-50 for Gram-positive bacteria and >100-125 for Gram-negative bacteria.

It has been found that with AUC/CMI<100 a 42% clinical and 26% microbiological efficiency may be obtained, and when ratio AUC/CMI is >125 response is usually 80% and 82% as to microbiological. Moreover, this may be increased by increasing the ratio, provided that toxicity is not caused.

Based on that above, it is apparent that pharmaceutical compositions are necessary to be generated allowing a rational use of antimicrobial drugs in commercial poultry and in pigs; that is, congruent with their PK/PD, for example, through suitable pharmaceutical designs for each antibacterial considering food and water consumption habits of commercial poultry and pigs. For example, it is known that enrofloxacin requires a strategic dosage for commercial poultry with a proper handling of water lines to promote an oral bolus dose in birds and thus provide key pharmacokinetic values for this antimicrobial drug considered as CD, reaching a maximum plasma concentration (Cmax) higher than 12 and a value of area under curve/minimum inhibiting concentration (AUC/CMI) higher than 125. When subject variables are not achieved, a lower clinical response is generated and resistant strain generation is promoted (Lenski, 1998). Management of water lines (Sumano et al., 2000; Dorrestein, 1991) and use of absorption promotings (Sumano & Gutiérrez 2003: Sumano et al., 2004) are actions which are congruent from PK/PD point of view for CD antibacterial drugs and optimize clinical response and productivity in a flock. Enrofloxacin and other fluoroquinolones shall not be administered by a single maneuver of adding to food since they do not achieve suitable Cmax values. This is even more critical in pigs and they must be individually injected thus involving management and cost. It has been noticed in pigs that oral route does not achieve a suitable F and therefore the proper Cmax is not reached, aside from a sick pig drastically reducing its feed and water intake when being sick.

The situation is critical for time-dependent (TD) antimicrobials as they are applied in feed and generally have short elimination half-lives (T½β) and often suitable therapeutic concentrations are not achieved by night, e.g., tylosin (Gutierrez et al., 2008). In spite of that, there are many DT antimicrobials used in poultry farming; for example: lincomycin and clindamycin, some tetracyclines, florphenicol and tianphenicol, tiamulin, phosphomycin and mixtures of sulfonamides with trimethoprim.

On the other hand, the precise pharmacokinetics of macrolides such as azithromycin, clarithromycin and roxithromycin is unknown in commercial poultry, which T½β is quite more extended in human beings (McConnell et al., 2006; Nightingale., 1997; Craig, 1997) and which may reach more favorable AUC/CMI variables, as well as 4 times MIC in all ID. However, that results less probable since drug excretion in birds is faster and bioavailability (F) is more reduced both by metabolic rate since almost one half of portal flow directly irrigates kidney (porta-renal) and this produces a remarkable effect of “first step” (Wages, 1997; Puyt, 1997). Furthermore, those macrolides should be assessed for use in poultry farming whether by cost and by being reserved in human medicine.

But above remarks are not only applicable to macrolides. It is not risky to say that most of poultry farming medicaments are bad designed; even poultry therapeutics icons such as oxytetracycline and chlortetracycline having a quite low bioavailability (F) (20%) and which have performed as antimicrobials with minor success given their potential.

There are antimicrobials, anticoccidial drugs, analgesics, mucolytics, vitamins, minerals and other medicaments with poorly suitable PK/PD designs within the state of the art; for example:

In commercial poultry:
Oxytetracycline with F of 20% which leads to dose use up to 1000 ppm.
Tylosin (phosphate or tartrate) in water or food with almost zero plasma concentrations by night and low F (<40%).
Phosphomycin in water which does not achieve high concentrations in usual doses (10-40 mg/kg/day) and which is removed from plasma and tissues in less than 6-8 hours.
Fluoroquinolones which even when not recommended for food medication, they are used in field with extremely low, frequently useless, Cmax results. Its F is 60% in drinking water in experimental models but a 180% F may be reached with this subject invention.
Erithromycin with a very low F (18%) and permanence in the organism which does not covers full day and much less by night.
   In pigs:
Mixtures of sulfonamides with trimethoprim do not have a synergic effect as in humans since trimethoprim is removed very fast (elimination half-life of 1 hr) and a minimum sulfonamide-trimethoprim 16:1 ratio is respectively required to meet synergy.
Lincomycin having a life of 20% or little more, thus limiting its clinical efficacy
Spectinomycin only has a 7% F and still synergistic with lincomycin for treatment of some diseases.
Tetracyclines require 800 ppm or higher doses to reach 40 mg/kg/day doses as their F is below 40%. Serum concentrations are not reached by night and in breeding sows doses should be 3000 ppm as they only eat an average of 2 kg with a weight of 300 kg. This is obviously not performed as they would reject food.
Tylosin and tiamulin are often underdosed due to costs. This fact limits efficacy, especially because they have a low F (not more than 50-60%) and given their fast elimination they show null concentrations by night.
The use of fluoroquinolones is currently NOT recommended in feed since high Cmax concentrations are not achieved (at least 12 times CMI) and/or AUC/CMI 125. FOLA system achieves that easily even better than if that would have been injected.
Provision of phosphomycin in food is not useful since its F is below 20% and is eliminated in a few hours. Associated to FOLA system the results allow a single dose in the morning with food reaching F close to 100% and during 24 hours.

In the light of above, the drawbacks shown by prior art prosthetic systems have been intended to be suppressed by developing a new composition of pharmaceutical excipients and forms allowing a remarkable increase in drug bioavailability in poultry, commercial poultry, as well as in pigs in every productive stage, optimizing its dosage and reducing antibacterial waste to the maximum, and further generating bacteria-resistant strains by optimizing drug pharmacokinetics/pharmacodynamics (PK/PD) ratio.

### OBJECTS OF INVENTION

Having in mind the deficiencies of prior art, it is an object of present invention to provide a composition of pharmaceutical forms and excipients to achieve an optimal bioavailability and long action sustained release (FOLA) of any drug in commercial poultry and pigs.

It is another object of present invention, to provide a composition of pharmaceutical forms and excipients to allow bioavailability (F) to be remarkably increased in antibacterial drugs, analgesics, mucolytics, anticoccidial drugs, vitamins and minerals

It is an additional object of present invention, to provide a composition of pharmaceutical forms and excipients allowing achievement of maximum response of drugs or added active substances, as they are more time available for absorption in gastrointestinal duct (GI), at a suitable rate to reach F maximum value while extending its permanence in the organism.

An additional object of present invention is to provide a composition of pharmaceutical forms and excipients administrable to broiler chicken, egg laying birds, egg breeding birds for production of broilers and “grandmothers” (parent breeding birds), ducks, turkeys, geese, quails, ostriches and other commercial poultry, as well as pigs in all productive stages, piglets, breeding stock, fatten, and others.

It is a further object of present invention, to provide a composition of pharmaceutical forms and excipients which optimizes dosage and reduces drug waste at maximum.

It is a further object of present invention, to provide a composition of pharmaceutical forms and excipients minimizing the generation of bacteria-resistant strains by optimization of pharmacokinetics/pharmacodynamics (PK/PD) ratio of antibacterial drugs and other drugs, such as NSAIDs, mucolytics, anticoccidial drugs, etc.

Still another further object of present invention is to provide a composition of pharmaceutical forms and excipients presented in several colors and shapes for identification and differentiation.

It is another object of present invention to provide a composition of pharmaceutical forms and excipients wherein the form which presents the composition allows birds to select pharmaceutical forms based on their instinct.

Still another further object of present invention is to provide a composition of pharmaceutical forms and excipients which masks flavor and odor.

### BRIEF DESCRIPTION OF FIGURES

Novel features which characterize present invention will be set forth in attached claims. However, the invention itself will be better understood, both in structural organization and in other objects and advantages thereof, by the following detailed description of certain preferred embodiments when reading together with the attached drawings wherein:

FIG. 1 shows a chart representing Enrofloxacin administered alone in food or included in FOLA system, in commercial poultry of 750 g±8.4 g, with ad-libitum food and estimating a dose of 8-12 mg/kg/day for food consumption. Note a generation of several enrofloxacin peaks with FOLA system, which makes suitable its PK/PD ratio.

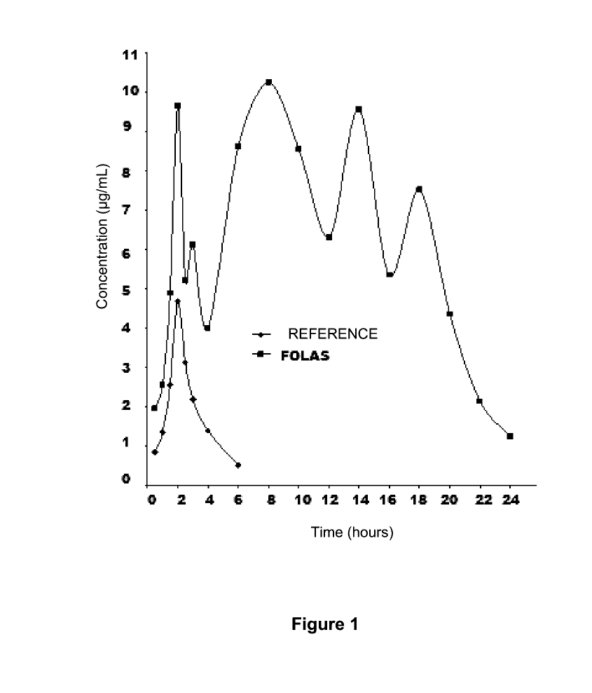

FIG. 2 shows a chart representing disodium phosphomycin administered alone in food or included in FOLA system, en commercial poultry de 750 g±10.2, with ad-libitum food and estimating a dose of 20 mg/kg/day for food consumption. Note a generation of two phosphomycin peaks with a higher AUC for the second peak with FOLA system, which makes suitable its PK/PD ratio.

FIG. 3 shows a chart representing Phosphomycin administered alone in food or included in FOLA system, in commercial poultry of 750 g±8.2, with ad-libitum food and estimating for food consumption a 40 mg/kg/day dose. Note a generation of two phosphomycin peaks with a larger AUC for the second peak with FOLA system, which makes suitable its PK/PD ratio.

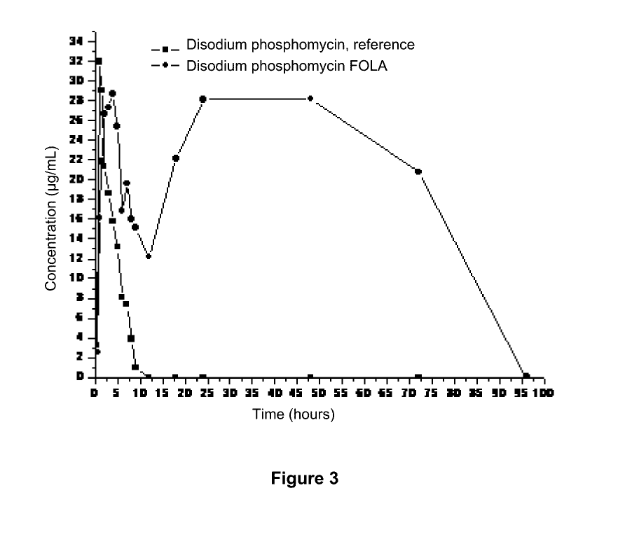

FIG. 4a shows a chart representing tylosin tartrate administered just in food or included in FOLA system, in commercial poultry of 1.9 kg±0.5, with ad-libitum food. Note generation of concentrations quite higher than traditional system (Cmax, MRT and AUC), which makes suitable its PK/PD ratio when included in FOLA system.

FIG. 4b shows a chart representing tylosin tartrate administered just in food or included in FOLA system, in commercial poultry of 1.9 kg±0.5, with ad-libitum food. Note generation of concentrations quite higher than traditional system, exceeding 1.5 μg/mL (Cmax, MRT and AUC), which makes suitable its PK/PD ratio when included in FOLA system.

FIG. 5 shows a chart representing serum concentrations of tiamulin fumarate along a day, administered just in food or included in FOLA system, in commercial poultry of 2.1 kg±0.6, with ad-libitum food. Note generation of concentrations quite higher than traditional system (Cmax, MRT and AUC), which makes suitable its PK/PD ratio when included in FOLA system.

FIG. 6 shows a chart representing tiamulin fumarate administered just in food or included in FOLA system during 6 days, in commercial poultry of 2.0 kg±0.6, with ad-libitum food. Note generation of concentrations quite higher than traditional system (Cmax, MRT and AUC), which makes suitable its PK/PD ratio when included in FOLA system.

FIG. 7 shows a chart representing serum concentrations of florphenicol along a day, administered just in food or included in FOLA system, in commercial poultry of 500 g±8, with ad-libitum food at 10 mg/kg. Note generation of concentrations higher than traditional system (MRT and AUC), which makes suitable its PK/PD ratio when included in FOLA system. It is remarked that Cmax is not pharmacologically significant for florphenicol.

FIG. 8a shows a chart representing serum concentrations of florphenicol along three days administered just in food or included in FOLA system, in commercial poultry of 450 g±9, with ad-libitum food at 20 mg/kg. Note generation of concentrations higher than traditional system (Cmax, MRT and AUC), which makes suitable its PK/PD ratio when included in FOLA system.

FIG. 8b shows a chart representing serum concentrations of florphenicol along three days administered just in food or included in FOLA system, in commercial poultry of 450 g±9, with ad-libitum food at 20 mg/kg. Note a generation of concentrations lower than 3 μg/mL, making suitable its PK/PD ratio when included in FOLA system.

FIG. 9 shows a chart representing serum concentrations of trimethoprim and sulfachloropyridazine sodium administered as premixture (5:1) (25 mg/kg of sulfonamide and 5 mg/kg of trimethoprim, in both cases) as conventionally in food or including active substances in FOLA system. Commercial poultry of 450 g±6, with ad-libitum food was used. Note generation of concentrations higher than traditional system (MRT and AUC), which makes suitable its PK/PD ratio when included in FOLA system. It is worth to remark that Cmax is not pharmacologically significant for this mixture, and a noteworthy feature of FOLA system is allowing a continued synergy during the whole dosage range and not only during the first 5 hours.

FIG. 10a shows a chart representing serum concentrations of oxytetracycline administered as premixture as conventionally in food or included in FOLA system. Commercial poultry of 700 g±6, with ad-libitum food was used and dosed at a 600 ppm rate. Note generation of concentrations higher than traditional system (Cmax, MRT and AUC), which makes suitable its PK/PD ratio when included in FOLA system. Note a significant improvement in bioavailability.

FIG. 10b shows a chart representing serum concentrations of oxytetracycline administered as premixture as conventionally in food or included in FOLA system related to any day hour. Commercial poultry of 700 g±6, with ad-libitum food was used and dosed at a 600 ppm rate. Note generation of concentrations higher than traditional system (Cmax, MRT and AUC), which makes suitable its PK/PD ratio when included in FOLA system. Note a significant improvement in bioavailability.

FIG. 11 shows a chart representing serum concentrations of 3 days of tilmicosin administered alone in food or included in FOLA system, in commercial poultry of 750 g±8, with ad-libitum food at a rate of 400 ppm. Note generation of concentrations higher than traditional system (Cmax, MRT and AUC) and a much more remarkable cumulative trend with FOLA system, which makes suitable its PK/PD ratio.

### DETAILED DESCRIPTION OF INVENTION

The present invention discloses compositions within a large variety of antimicrobials, anticoccidial drugs, analgesics, vitamins, mucolytics, minerals and other drugs by manipulation of the pharmaceutical form and excipients used for notoriously increasing their bioavailability (F), frequently their Cmax and to extend their duration or permanence in bird and pig organisms, with an optimal pharmacokinetics (drug destination in bird's organism) with pharmacodynamics (mechanism whereby they exert their effect at cell or tissue level) ratio (PK/PD) and which results in better clinical efficacy in each medicament.

Compositions subject of present invention comprise the form, composition, size and color of solid shapes which contribute for selection and consumption by birds and pigs, since the medicament is usually mixed with food. For example, in poultry such as hens, they tend to select those foods with shapes equivalent to cereal grains, worms and other organic forms and specific colors. As to pigs, flavor is masked thus leading to a better acceptance and a remarkable increase of F in antibacterial drugs, analgesics, mucolytics, anticoccidial drugs, beta-adrenergic agonists such as ractopamin, vitamins and minerals.

The compositions subject of present invention comprise:
about 10 to 70% of one or more pharmaceutically active agents, selected from the group comprising: antimicrobials, anticoccidial drugs, analgesics, vitamins, mucolytics, minerals, and others;
about 0.5 to 20% of one or more bioavailability promoting agents, selected from the group consisting of capsaicin and derivatives, grapefruit and its extracts, cyclodextrines, labrasol, sodium caproate (0.25%) sodium desoxycholate (1.0%), hexadecyldimethylbenzylammonium chloride, hexylsalicylic acid, polyacrylic acid cysteine/glutathione reduced of chitosan-4-thio-butylamide (chitosan-TBA)/reduced glutathione, EDTA and TRIS.
about 20 to 80% of one or more polymers for drug sustained release selected from the group consisting of poloxamer, carbopol, methocel, β-cyclodextrin, poli (D, L lactide) (PDLA), poli (L-lactide) (PLLA), tragacanth gum (high concentration), guar gum, karaya gum (high concentration), sodium alginate, gelatin, chitosan; cellulose derivatives such as methylcellulose (low molecular weight), sodium carboxymethylcellulose (low, medium and high molecular weight), hydroxyethyl-cellulose, hydroxypropylcellulose, poliethyleneglycols (high molecular weight), polyvinyl alcohol, carbopol, acrylic and methacrylic acid polymers and copolymers, polyalkylcyanoacrylates, polycarbophil, polyacrylic acid, sodium alginate and hydroxypropylmethylcellulose, carbopol 934 and EX55, carragenate, guar gum, methylcellulose 10 cPs, polyacrylamide, polycarbophil, tragacanth, polyacrylic acid crosslinked with sucrose, polymethacrylic acid, carbopol base with petroleum jelly/hydrophilic paraffin, katara gum, acacia, alginic acid, agar-agar, pectin and amilopectin, calcium carboxymethylcellulose, polyhydroxyethylmethacrylate (PHEMA), methylcellulose, higher than 100 cPs, polyvinylpyrrolidone, degraded carragenate, dextrans and other polymers;
about 0.01 to 1% of one or more food grade or animal consumption colorants; and
about 0.1 to 5% of natural or artificial flavorants.

This composition is mixed and extruded to provide shape and appearance allowing a better acceptance by any bird or pig.

The compositions subject of present invention are also characterized by comprising drugs preferably selected from the group of time-dependent antimicrobials but also some concentration-dependent drugs such as: tylosin, tiamulin, tilmicosin, enrofloxacin and other fluoroquinolones, phosphomycin, florphenicol, oxytetracycline, doxycycline, erithromycin and other macrolides, clortetracycline, sulfonamides with trimethoprim, and others.

The compositions subject of present invention, preferably comprise those colorants corresponding to red, yellow, green and orange hues, as well as their combinations. Shapes which the compositions subject of present invention are extruded into are varied, including spheres, cylinders, flat or cylindrical worms, straight or curved, coiled, irregular flat or filled shapes, etc.

The compositions of excipients and pharmaceutical forms with sustained release and increase in drug bioavailability are prepared by following the procedure described below:

Drug or active substance is dry mixed with the bioavailability promoting agent(s), one or more agents destined to achieve sustained release or long action are then added. These ingredients are mixed until homogenizing the mixture, and adding colorants or flavorants as required. Once a homogeneous mixture is achieved, from 10 to 60% by weight of the water total mixture is added, mixing until obtaining a mass of dry to semi-dry and soft consistency.

The soft and dried mass is poured into extrusion equipment, its nozzle being adapted with the physical shape of the above mentioned selected pharmaceutical shape. Extruded fragments are dried at room temperature, protected from light and air.

Obtained product is the composition of pharmaceutical forms and excipients with sustained release and increase in drug bioavailability for poultry and pigs which optimizes drug dosage and reduces waste thereof; minimizes generation of bacteria-resistant strains by optimizing pharmacokinetics/pharmacodynamics ratio in drugs and further masking drug flavor and odor.

Compositions of pharmaceutical excipients and forms were prepared by above procedure, which were tested in birds and pigs, according to the examples described below. The present invention will be better understood from the following examples which are only provided for illustrative purposes allowing a full understanding of the preferred embodiments of present invention, not excluding that there are other non-illustrated embodiments which may be practiced based on above disclosed detailed description.

Examples below are described illustratively but not limiting the scope of the invention.

### Examples

### Tests in Commercial Poultry

### Example 1

### Enrofloxacin

As to enrofloxacin, a fluoroquinolone which according to worldwide standards SHALL NOT be administered in food and nevertheless, an exceptional pharmacokinetics is achieved with FOLA-enrofloxacin, better than any known fluoroquinolone at this date. Data are revealing a unique therapeutic potential as shown below.

10 grams of enrofloxacin

20 grams of methocel

30 grams of wheat flour

1 mg of a food grade green colorant, following the previously described procedure.

Pharmaceutical form was obtained in the form of a little stone as irregular spheres:

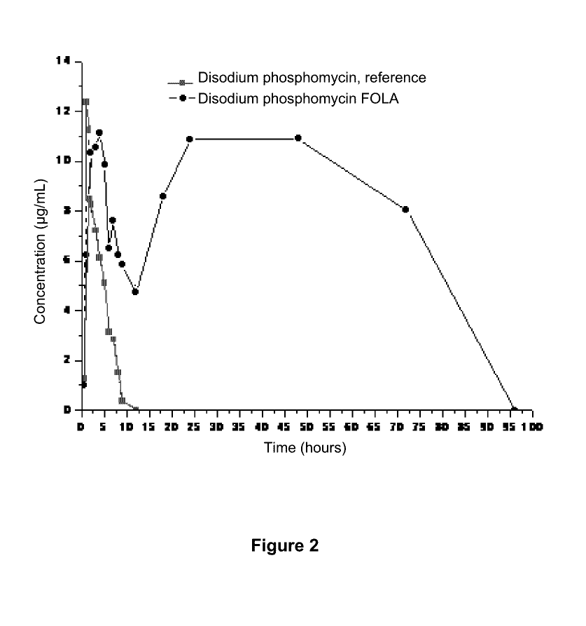

Single doses of 10 mg/kg were administered in food ad libitum* or in drinking water **, obtaining the results which are illustrated in FIG. 2, chart 1, comparing with the results obtained by administering commercially available enrofloxacin.

| | Dose in | | | AUC/CMI | Cmax/CMI | |
|---|---|---|---|---|---|---|
| | food* or | AUC | Cmax | (w/o | (w/o | Fr |
| Drug | in water** | (μg/mL/h) | (μg/mL) | units) | units) | (%) |
| Enrofloxacin | 10 mg/kg* | 156.3 | 10.23 | 2605 | 170.5 | 1766 |
| in FOLA | | | | | | |
| Enrofloxacin | 10 mg/kg** | 8.85 | 4.2 | 147 | 70 | 100 |
| commercially | | | | | | |
| available | | | | | | |

AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case E. coli (0.06 μg/mL).
Cmax/CMI = valuw which must be 10-12 or above if possible in fluoroquinolones.
Fr = Relative bioavailability achieved with formula
AUC_{FOLA}/AUC_{reference} × 100.
*Estimating a daily food consumption according to bird age

### Example 2

### Phosphomycin

Following is detailed a manufacturing procedure of FOLA-disodium phosphomycin as disodium phosphomycin is a very common antibacterial drug in Latin America, and data from two assays are presented wherein a huge difference is apparent between a F achieved with commercially obtained disodium phosphomycin reference premixture and that achieved with FOLA system, in these cases using a dose of 20 and 40 mg/kg/day in food ad libitum with both preparations.

A composition was prepared by mixing 3 grams of phosphomycin, about 0.5 grams of Methocel, about 6.5 grams of wheat flour and 5 mg of food grade green colorant, extruding this mixture in spherical forms.

Variables disclosed below are pharmacokinetically obtained for disodium phosphomycin in FOLA and a commercially available reference preparation. Charts 2A and 2B illustrate the obtained results.

Single dose of 20 mg/kg in food ad libitum*

| | Dose in | AUC | Cmax | AUC/CMI | Fr |
|---|---|---|---|---|---|
| Drug | food* | (μg/mL/h) | (μg/mL) | (w/o units) | (%) |
| Disodium | 10 mg/kg | 714.04 | 11.36 | 7140.4 | |
| phosphomycin | | | | | |
| en FOLA | | | | | |
| Disodium | 10 mg/kg | 42.17 | 11.15 | 421.7 | |
| phosphomycin | | | | | |
| commercially | | | | | |
| available | | | | | |

Single dose of 40 mg/kg in food ad libitum*

| | Dose in | AUC | Cmax | AUC/CMI |
|---|---|---|---|---|
| Drug | food* | (μg/mL/h) | (μg/mL) | (w/o units) |
| Disodium | 10 mg/kg | 1422.12 | 23.58 | 14221.2 |
| phosphomycin | | | | |
| en FOLA | | | | |
| Disodium | 10 mg/kg | 83.26 | 21.36 | 832.6 |
| phosphomycin | | | | |
| commercially | | | | |
| available | | | | |

AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case Haemophilus gallinarum (0.1-0.4 μg/mL).
Fr = Relative bioavailability achieved with formula AUC_{FOLA}/AUC_{reference} × 100.
*Estimating a daily food consumption according to bird age

Examples with Other Antibacterial Drugs in Birds and Pigs

Compositions of excipients and pharmaceutical forms with sustained release and increase in bioavailability were similarly prepared with oxytetracycline, florphenicol, tilmicosin, tylosin, tiamulin, and sulfachloropyridazine sodium with trimethoprim to be administered in birds; compositions were prepared for administration to pigs by using about 30% by weight of drug; about 5% of one or more bioavailability promoting agents, selected from the group consisting of capsaicin, grapefruit extracts, cyclodextrins, labrasol, sodium caprate (SC, 0.25% w/v, sodium desoxycholate (SD, 1.0% w/v), hexadecyldimethylbenzylammonium chloride, hexylsalicylic acid, polyacrylic acid cysteine/glutathione reduced of chitosan-4-thio-butylamide (chitosan-TBA)/reduced glutathione, EDTA and TRIS; about 65% of one or more drug sustained-release polymers selected from the group consisting of poloxamer, carbopol, methocel, β-cyclodextrin, poly (D, L lactide) (PDLA), poly (L lactide) (PLLA), tragacanth gum (high concentration), guar gum, karaya gum (high concentration), sodium alginate, gelatin, chitosan; cellulose derivatives such as methylcellulose (low molecular weight), sodium carboxymethylcellulose (high molecular weight), hydroxyethyl cellulose, hydroxypropylcellulose, polyethylene glycols (high molecular weight), polyvinyl alcohol, carbopol, acrylic and methacrylic acid polymers and copolymers, polyalkylcyanoacrylates, polycarbophil, chitosan, polyacrylic acid, Sodium alginate, Carbopol and hydroxypropylmethylcellulose, Carbopol 934 and EX55, Sodium carboxymethylcellulose, Carragenate, Guar gum, Hydroxyethyl cellulose, Methyl cellulose 10 cPs, Polyacrylamide, Polycarbophil, Tragacanth, Sucrose-crosslinked polyacrylic acid, polymethacrylic acid, Carbopol base with petroleum jelly/hydrophilic paraffin, katara gum, Hydroxypropyl cellulose, Acacia, alginic acid, Agar-agar, Amilopectin, Calcium carboxymethylcellulose, Polyhydroxyethylmethacrylate (PHEMA), Methylcellulose, higher than 100 cPs, Pectin, Polyethylene glycol, Polyvinylpyrrolidone, degraded Carragenate, and Dextrans; and about 0.5% of a colorant selected from the group which tones are red, orange, Green and yellow; extruded in shapes of cylinders, spheres, irregular spheres, worms, screws, and others.

Obtained results are shown below:

### Example 3

### Tylosin

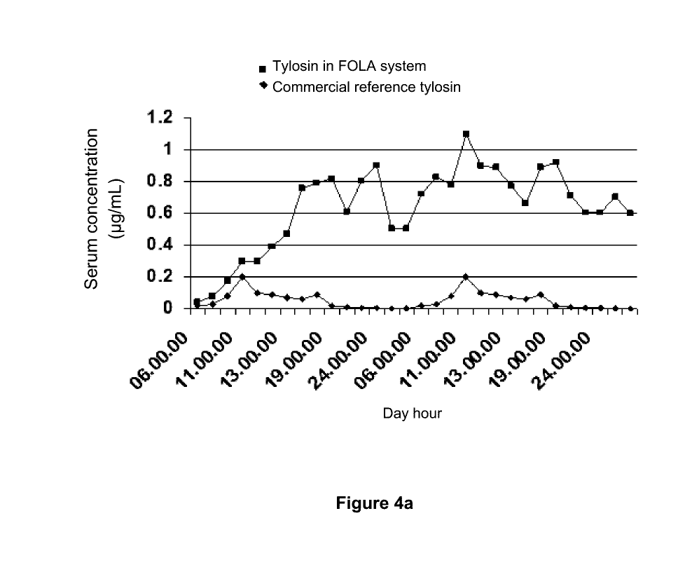

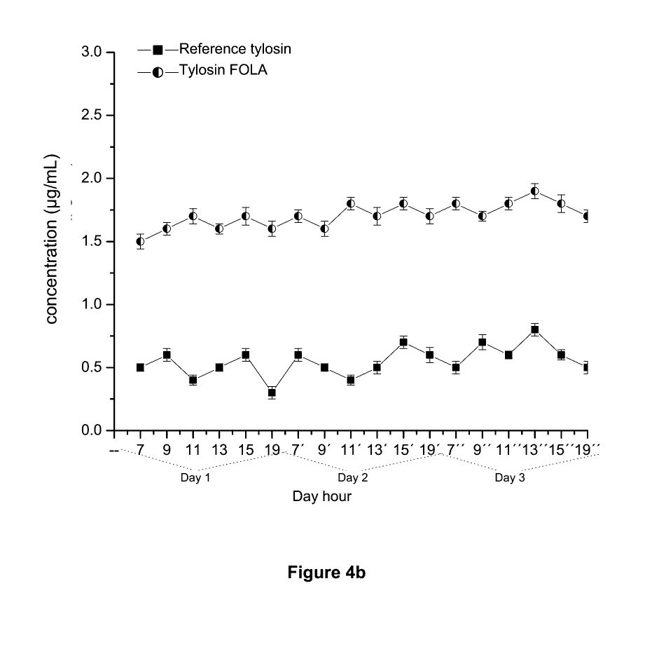

For Tylosin in commercial poultry, very high concentrations are achieved when FOLA system is used and which remain with therapeutic effect along night as shown in charts of FIGS. 4a and 4b. Variables disclosed below are pharmacokinetically obtained for tylosin in FOLA and a commercially available reference preparation:

| | Dose in | AUC | Cmax | AUC/CMI | Fr |
|---|---|---|---|---|---|
| Drug | food | (μg/mL/h) | (μg/mL) | (w/o units) | (%) |
| Tylosin | 200 ppm | 18.4 | 1.13 | 184 | 868 |
| tartrate | | | | | |
| in FOLA | | | | | |
| Tylosin | 200 ppm | 2.12 | 0.2 | 2.0 | 100 |
| tartrate | | | | | |
| commercially | | | | | |
| available | | | | | |

AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case Mycoplasma spp (0.1 μg/mL).
Fr = Relative bioavailability achieved with formula AUC_{FOLA}/AUC_{reference} × 100

### Example 4

### Tiamulin

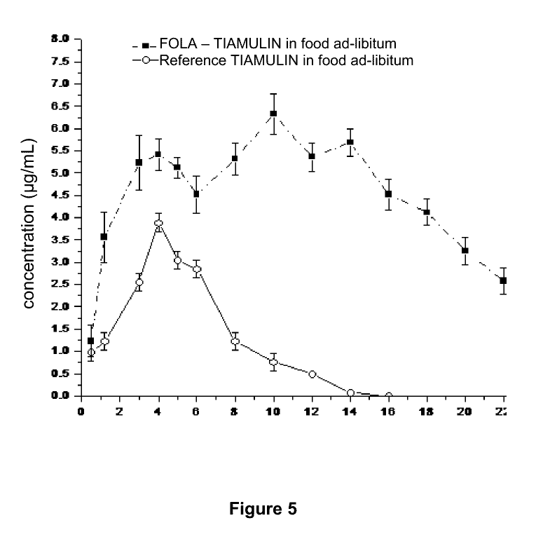

The following representative results are shown for tiamulin, as illustrated in the chart of FIG. 5. Variables disclosed below are pharmacokinetically obtained for tiamulin in FOLA and a commercially available reference preparation:

Dose of 50 mg/kg/day per 6 days, ad libitum*.

| | Dose in | AUC | Cmax | AUC/CMI | Fr |
|---|---|---|---|---|---|
| Drug | food* | (μg/mL/h) | (μg/mL) | (w/o units) | (%) |
| Tiamulin | 50 mg/kg | 93.75 | 6.25 | 937.5 | 535.71 |
| en FOLA | | | | | |
| Tiamulin | 50 mg/kg | 17.50 | 3.81 | 175.0 | 100 |
| commercially | | | | | |
| available | | | | | |

AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case Mycoplasma spp (0.6-1.5 μg/mL for sensitive microorganisms and de 1.5 a 2.6 μg/mL).
Fr = Relative bioavailability achieved with formula AUC_{FOLA}/AUC_{reference} × 100.
*Estimating a daily food consumption according to bird age

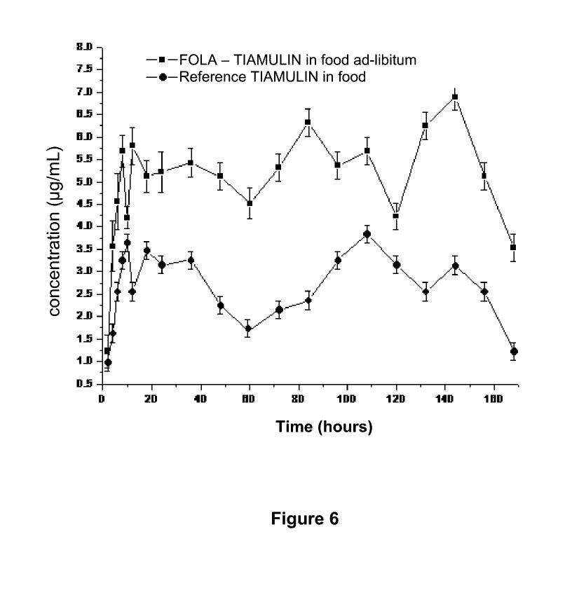

When the study is extended by 6 days, the medication provides the results shown in the chart of FIG. 6. Variables disclosed below are pharmacokinetically obtained for tiamulin en FOLA and a commercially available reference preparation:

Single dose of 50 mg/kg in food ad libitum*

| | Dose in | AUC | Cmax | AUC/CMI | Fr |
|---|---|---|---|---|---|
| Drug | food* | (μg/mL/h) | (μg/mL) | (w/o units) | (%) |
| Tiamulin | 50 mg/kg | 104.2 | 6.82 | 69.46 | 268.4 |
| in FOLA | | | | | |
| Tiamulin | 50 mg/kg | 52.9 | 3.84 | 35.26 | 100 |
| commercially | | | | | |
| available | | | | | |

AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case Mycoplasma spp (0.6-1.5 μg/mL for sensitive microorganisms and de 1.5 a 2.6 μg/mL)
Fr = Relative bioavailability achieved with formula AUC_{FOLA}/AUC_{reference} × 100
*Estimating a daily food consumption according to bird age

### Example 5

### Florphenicol

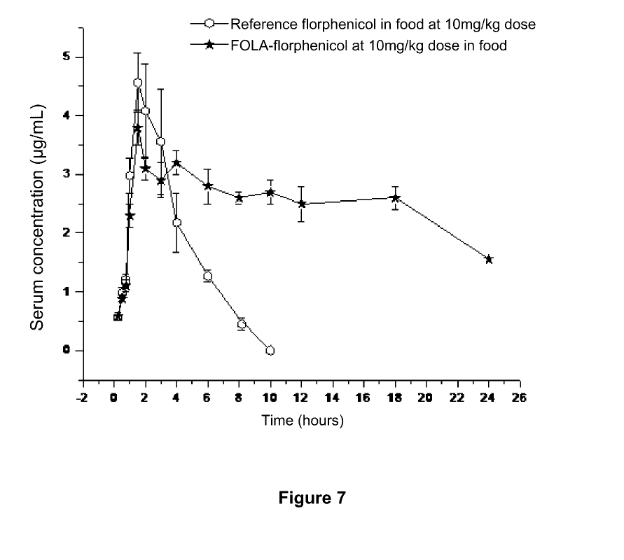

Multiple assays were conducted for florphenicol using doses of 10 mg/kg in food, with estimated daily food consumption according to bird age. Results are shown in the chart of FIG. 7. Variables disclosed below are pharmacokinetically obtained for florphenicol in FOLA and a commercially available reference preparation:

Single dose of 10 mg/kg in food ad libitum*

| | Dose in | AUC | Cmax | AUC/CMI | Fr |
|---|---|---|---|---|---|
| Drug | food* | (μg/mL/h) | (μg/mL) | (w/o units) | (%) |
| Florphenicol | 10 mg/kg | 38.15 | 3.8 | 381.5 | 244.55 |
| in FOLA | | | | | |
| Florphenicol | 10 mg/kg | 15.6 | 4.5 | 156.0 | 100 |
| commercially | | | | | |
| available | | | | | |

AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case Haemophilus gallinarum (0.1-0.4 μg/mL).
Fr = Relative bioavailability achieved with formula AUC_{FOLA}/AUC_{reference} × 100.
*Estimating a daily food consumption according to bird age

### Example 6

### Sulfachloropyridazine Na Con Trimethoprim

It is worth to remark that a synergy as that expected in vivo is not achieved under normal conditions for a mixture of sulfachloropyridazine Na with trimethoprim. This is due to a fast elimination of trimethoprim in birds (T½β=1 hour vs. T½β in humans=10 hours). A true synergy is achieved with FOLA system while keeping concentrations of at least 16-20 parts of sulfonamide per 1 of trimethoprim during the whole dosage range, this ratio being necessary to keep synergy.

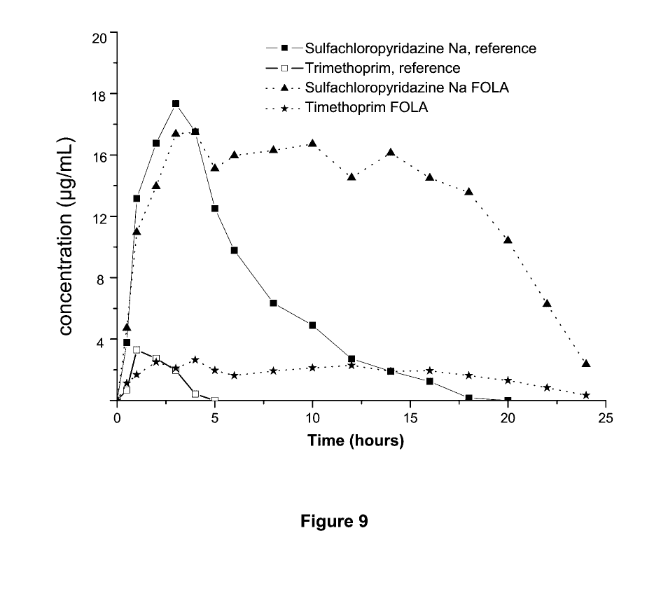

The chart in FIG. 9 presents the constant in sulfachloropyridazine+trimethoprim concentrations in (5:1) ratio and dosed together at a rate of 25 mg/kg of sulfonamide and 5 mg/kg of trimethoprim in food and using a commercial preparation as reference. Assessment of sulfonamide and trimethoprim concentrations was made by high resolution liquid chromatography. Obtained results are shown below in quadruplicate.

Variables disclosed below are pharmacokinetically obtained for sulfachloropyridazine Na and trimethoprim in FOLA and with a commercially available reference preparation:

Single dose of 25 mg/kg de sulfachloropyridazine Na and 5 mg/kg de trimethoprim in food, ad libitum*

SULFACHLOROPYRIDAZINE - Na

| | Dose | | | AUC/CMI | | SCP- |
|---|---|---|---|---|---|---|
| | in | AUC | Cmax | (w/o | Fr | Na/TMP |
| Drug | food* | (μg/mL/h) | (μg/mL) | units) | (%) | Ratio |
| Sulfachloropyridazine | 20 mg/kg | 241.4 | 16.8 | 60.35 | 226 | 5.5/1 |
| Na | de | | | | | |
| in FOLA | SCP-Na | | | | | |
| | and 4 mg/kg | | | | | |
| | de TMP | | | | | |
| Sulfachloropyridazine | 20 mg/kg | 107.5 | 17.3 | 26.75 | 100 | 4.8/1 |
| Na, | de | | | | | |
| commercially | SCP-Na | | | | | |
| available | and 4 mg/kg | | | | | |
| preparation | de TMP | | | | | |

TRIMETHOPRIM

| | Dose in | AUC | Cmax | Fr |
|---|---|---|---|---|
| Drug | food* | (μg/mL/h) | (μg/mL) | (%) |
| Trimethoprim | 4 mg/kg | 54.35 | 2.9 | 610.7 |
| in FOLA | de SCP-Na | | | |
| | and 4 mg/kg | | | |
| | de TMP | | | |
| Trimethoprim | 4 mg/kg | 8.9 | 3.6 | 100 |
| of a commercially | de SCP-Na | | | |
| available | and 4 mg/kg | | | |
| preparation | de TMP | | | |

SCP-Na = sulfachloropyridazine Na
TMP = trimethoprim
AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case E. coli (4-10 μg/mL para SCP-Na and de 1-2 para TMP and de 0.4-1 para la acción conjunta de SCP-Na con TMP).
Fr = Relative bioavailability achieved with formula AUC_{FOLA}/AUC_{reference} × 100.
*Estimating a daily food consumption according to bird age

### Example 7

### Oxytetracycline

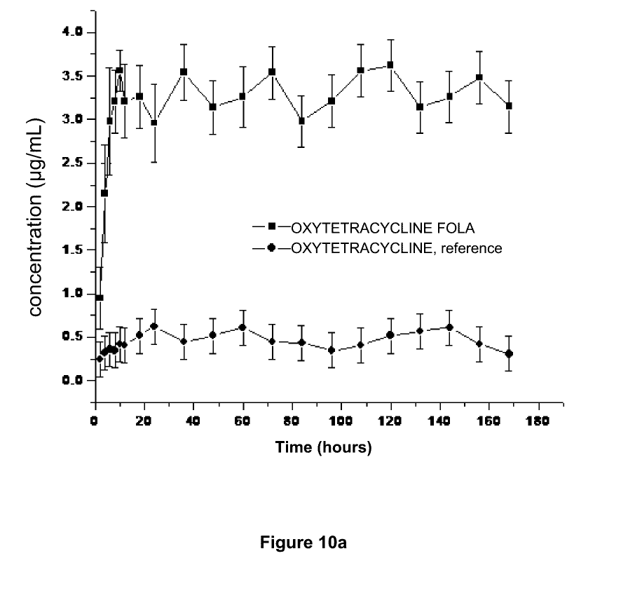

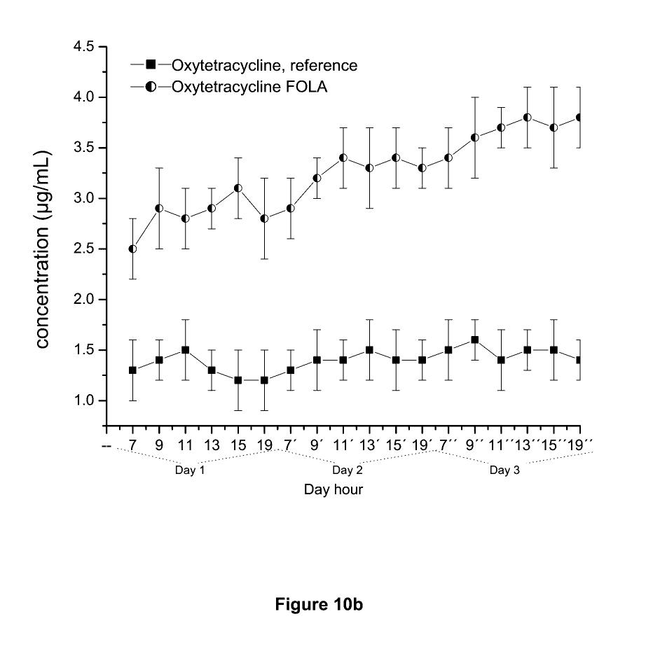

Oxytetracycline has been and still remains as the most successful antibacterial drug ever sold in the history of animal production (birds and pigs), administered as premixture; however, oxytetracycline base bioavailability in chickens and commercial poultry fluctuates around 20%. In such a way that high concentrations in food must be used for a minimum effect and somehow questionable concentrations when considering that CMI for E. coli is 2.5 μg/mL. As noticed, unprecedented serum concentrations with FOLA system at a 600 ppm dose are achieved, and thus PK/PD congruence for an antibacterial drug having been irrationally used for more than half a century. Obtained results are shown in charts from FIGS. 10a and 10b.

Variables disclosed below are pharmacokinetically obtained for oxytetracycline in FOLA and a commercially available reference preparation:

Dose of 600 ppm in food ad libitum*

| | Dose in | AUC | Cmax | AUC/CMI | Fr |
|---|---|---|---|---|---|
| Drug | food* | (μg/Ml/h) | (μg/mL) | (w/o units) | (%) |
| Oxytetracycline | 600 ppm | 659.45 | 0.6 | 6594.5 | 705.89 |
| in FOLA | | | | | |
| Oxytetracycline | 600 ppm | 93.42 | 3.6 | 934.2 | 100 |
| commercially | | | | | |
| available | | | | | |

AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case Haemophilus gallinarum (0.1-0.4 μg/mL).
Fr = Relative bioavailability achieved with formula AUC_{FOLA}/AUC_{reference} × 100.
*Estimating a daily food consumption according to bird age

### Tests in Birds and Pigs

### Example 8

### Oxytetracycline

Chart 8 shows obtained results. Variables disclosed below are pharmacokinetically obtained for oxytetracycline in FOLA and a commercially available reference preparation administered during 3 days:

Single dose of 1200 ppm in food ad libitum*

| | Dose in | AUC | Cmax | AUC/CMI | Fr |
|---|---|---|---|---|---|
| Drug | food* | (μg/Ml/h) | (μg/mL) | (w/o units) | (%) |
| Oxytetracycline | 1200 | 168.9 | 3.84 | | 232.8 |
| in FOLA | ppm | | | | |
| Oxytetracycline | 1200 | 72.6 | 1.62 | | 100.00 |
| commercially | ppm | | | | |
| available | | | | | |

AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case Haemophilus suis (0.1-0.4 μg/mL).
Fr = Relative bioavailability achieved with formula AUC_{FOLA}/AUC_{reference} × 100.
*Estimating a daily food consumption according to pig age

### Tests in Commercial Poultry

### Example 9

### Florphenicol

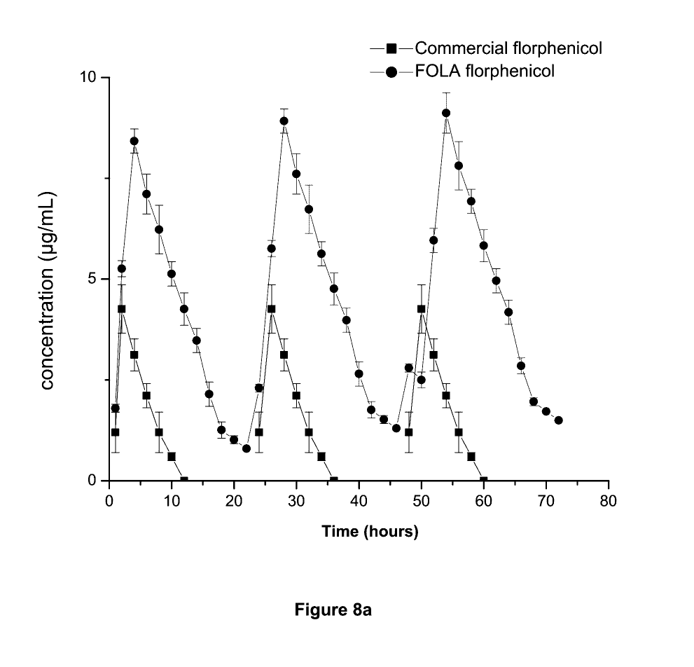

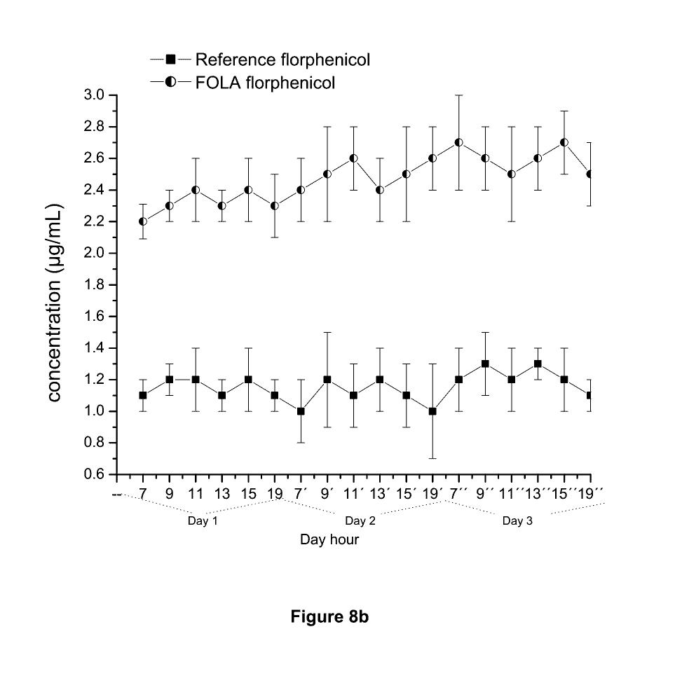

Chart in FIG. 8b shows obtained results. Variables disclosed below are pharmacokinetically obtained for florphenicol in FOLA and a commercially available reference preparation administered during 3 days:

Single dose of 20 mg/kg in food ad libitum*

| | Dose in | AUC | Cmax | AUC/CMI | Fr |
|---|---|---|---|---|---|
| Drug | food* | (μg/Ml/h) | (μg/mL) | (w/o units) | (%) |
| Florphenicol | 20 mg/kg | 130.1 | 2.76 | | 220 |
| FOLA | | | | | |
| Florphenicol | 20 mg/kg | 59.15 | 1.31 | | 100 |
| commercially | | | | | |
| available | | | | | |

AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case Haemophilus suis (0.1-0.4 μg/mL).
Fr = Relative bioavailability achieved with formula AUC_{FOLA}/AUC_{reference} × 100.
*Estimating a daily food consumption according to pig age

### Example 10

### Tilmicosin

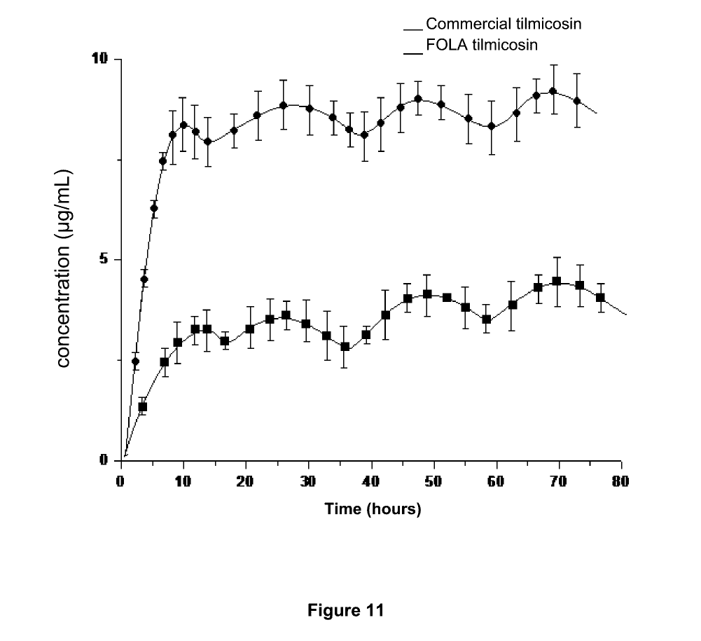

Chart in FIG. 11 shows obtained results. Variables disclosed below are pharmacokinetically obtained for Tilmicosin in FOLA and a commercially available reference preparation administered during 3 days:

Multiple dose of Tilmicosin 400 ppm in food ad libitum*

| | Dose in | AUC | Cmax | AUC/CMI | Fr |
|---|---|---|---|---|---|
| Drug | food* | (μg/Ml/h) | (μg/mL) | (w/o units) | (%) |
| Tilmicosin | 20 mg/kg | 89.3 | 1.92 | | 311.7 |
| FOLA | | | | | |
| Tilmicosin | 20 mg/kg | 28.65 | | | 100 |
| commercially | | | | | |
| available | | | | | |

AUC = area under curve of drug concentration vs time with FOLA system or with the commercially available preparation (reference).
Cmax = maximum serum or plasma concentration
AUC/CMI = ratio between AUC value divided by minimum inhibitory concentration of a pathogen, in this case Haemophilus suis (0.1-0.4 μg/mL).
Fr = Relative bioavailability achieved with formula AUC_{FOLA}/AUC_{reference} × 100.
*Estimating a daily food consumption according to pig age

Even though certain embodiments of the invention have been illustrated and described, it should be noted that a number of modifications thereof are possible, but such modifications do not represent a distance from the true scope of the invention. Therefore, the present invention shall not be considered restricted except by the provisions in the state of the art, as well as the scope of the attached claims.

### REFERENCES

1. Craig W A. Antimicrobial resistance issues of the future. Diagn Microbial Infect Dis. 1996; 25:213-217.
2. Dorrestein, G. M. and A. S J. P A. M. Van Miert. Pharmacotherapeutic aspects of medication of birds. J Vet Pharmacol Ther 1988. 11:33-44.
3. Dorrestein, G. M. The pharmacokinetics of avian therapeutics. Vet Clin North Am Small Anim Pract 1991. 21:1241-1261.
4. Pigan, D. and R, Kolter. 2002. Why are bacteria refractory to antimicrobials. Curr. Opin. Micro. 5:472-477.
5. Lenski, R. E. (1998). The cost of antibiotic resistance—from the perspective of a bacterium. Ciba Found Symp 207, 131-140.
6. McConell J, 2006, New Orraly active carbapanem trial results announced. Lancet Infect Dis. 6: 265-266
7. Nightingale C. H., Pharmacokinetics and pharmacodynamics of newer macrolides. Pediatr. Infect. Dis. J. 16 (1997), pp. 438-443.
8. Puyt J. D., Antibiotic therapy in poultry production. Bulletin des Groupments Techniques Vétérinaires 5 (1995), pp. 17-110
9. Sumano L. H, & Gutierrez O. L. (2003) Strategic administration of enrofloxacin in poultry to achieve higher maximal serum concentrations. The Veterinary Journal. 165, 143-148.
10. Sumano L. H, Gutierrez O. L & Ocampo C. L. (2006) Bioequivalence comparison of seventeen commercial oral enrofloxacins against the original preparation in broilers. Journal of Poultry Science. 43, 23-28.
11. Sumano L. H. & Gutierrez, O. L. (2000). Problemática del use de la enrofloxacin en la avicultura en México. Veterinaria México, 31, 137-145.
12. Sumano L. H., Gutierrez O. L. & Zamora Q. M. A. (2000). Bioequivalence of six trademarks of enrofloxacin in poultry. Journal of Veterinary Pharmacology and Therapeutics. 24, 1-5.
13. Sumano L. H., Gutierrez O. L., Aguilera R., Rosiles, M. R., Bernad B. M. J. & Gracia M. J. (2004). Influence of hard water on the bioavailability of enrofloxacin in broilers. Poultry Science. 83, 726-781.
14. Wages, D. P. (1997). Proper medication procedures. Poultry Digest. 56

1. A composition of pharmaceutical forms and excipients with sustained release and increase in drug bioavailability for poultry and pigs, characterized in that comprises: a) about 10 to 70% of one or more pharmaceutically active agents, b) about 0.5 to 20% of one or more bioavailability promoting agent(s), c) about 20 to 80% of one or more drug sustained-release polymers, d) about 0.01 to 1% of one or more colorants, and d) about 0.1 to 5% of one or more flavorants.

2. The composition of pharmaceutical forms and excipients with sustained release and increase in drug bioavailability for poultry and pigs according to claim 1, characterized in that the pharmaceutically active agent is selected from the group comprising: antimicrobials, anticoccidial drugs, analgesics, vitamins, mucolytics and minerals, and the like.

3. The composition of pharmaceutical forms and excipients with sustained release and increase in drug bioavailability for poultry and pigs according to claim 2, characterized in that said antimicrobial is selected from the group comprising: tylosin, tiamulin, tilmicosin, enrofloxacin and other fluoroquinolones, phosphomycin, florphenicol, oxytetracycline, doxycycline, erithromycin and other macrolides, chlortetracycline, sulfonamides with trimethoprim, and the like.

4. The composition of pharmaceutical forms and excipients with sustained release and increase in drug bioavailability for poultry and pigs according to claim 1, characterized in that the bioavailability promoting agent is selected from the group comprising: capsaicin and derivatives, grapefruit and its extracts, cyclodextrins, labrasol, sodium caproate (0.25%), sodium desoxycholate hexadecyldimethylbenzylammonium chloride, hexylsalicylic acid, polyacrylic acid cysteine/glutathione reduced of chitosan-4-thio-butylamide (chitosan-TBA)/reduced glutathione, EDTA and TRIS.

5. The composition of pharmaceutical forms and excipients with sustained release and increase in drug bioavailability for poultry and pigs according to claim 1, characterized in that the drug sustained-release polymer is selected from the group comprising poloxamer, carbopol, methocel, β-cyclodextrin, poly(D, L lactide) (PDLA), poly(L-lactide) (PLLA), tragacanth gum (high concentration), guar gum, karaya gum (high concentration), sodium alginate, gelatin, chitosan; cellulose derivatives such as methylcellulose (low molecular weight), sodium carboxymethylcellulose (low, medium and high molecular weight), hydroxyethyl-cellulose, hydroxypropylcellulose, polyethylene glycols (high molecular weight), polyvinyl alcohol, carbopol, acrylic and methacrylic acid polymers and copolymers, polyalkylcyanoacrylates, polycarbophil, polyacrylic acid, sodium alginate and hydroxypropylmethylcellulose, carbopol 934 and EX55, carragenate, guar gum, methylcellulose 10 cPs, polyacrylamide, polycarbophil, tragacanth, polyacrylic acid crosslinked with sucrose, polymethacrylic acid, carbopol base with petroleum jelly/hydrophilic paraffin, katara gum, acacia, alginic acid, agar-agar, pectin and amilopectin, calcium carboxymethylcellulose, polyhydroxyethylmethacrylate (PHEMA), methylcellulose, higher than 100 cPs, polyvinylpyrrolidone, degraded carragenate and dextrans, and the like.

6. The composition of pharmaceutical forms and excipients with sustained release and increase in drug bioavailability for poultry and pigs according to claim 1, characterized in that the colorant is food grade or animal consumption colorant.

7. The composition of pharmaceutical forms and excipients with sustained release and increase in drug bioavailability for poultry and pigs according to claim 6, characterized in that the colorant corresponds to red, yellow, green and orange hues, as well as their combinations.

8. The composition of pharmaceutical forms and excipients with sustained release and increase in drug bioavailability for poultry and pigs according to claim 1, characterized in that the flavorant is natural or artificial.

9. The composition of pharmaceutical forms and excipients with sustained release and increase in drug bioavailability for poultry and pigs according to claim 1, characterized in that optimizes drug dosage and reduces waste thereof.

10. The composition of pharmaceutical forms and excipients with sustained release and increase in drug bioavailability for poultry and pigs according to claim 1, characterized in that bacteria-resistant strain generation is minimized by optimization of the pharmacokinetics/pharmacodynamics ratio of drugs.

11. The composition of pharmaceutical forms and excipients with sustained release and increase in drug bioavailability for poultry and pigs according to claim 1, characterized in that drug flavor and odor is masked.

12. The composition of pharmaceutical forms and excipients with sustained release and increase in drug bioavailability for poultry and pigs according to claim 1, characterized in that is indicated for administration in birds selected from the group comprising: broiler chicken, egg laying birds, egg breeding birds, broilers, parent breeding birds, ducks, turkeys, geese, quails and ostriches and the like.

13. The composition of pharmaceutical forms and excipients with sustained release and increase in drug bioavailability for poultry and pigs according to claim 1, characterized in that is indicated for administration to pigs in any productive stage: piglets, breeding stock, fatten and others.

14. The composition of pharmaceutical forms and excipients with sustained release and increase in drug bioavailability for poultry and pigs according to claim 1, characterized in shapes such as: spheres, cylinders, flat or cylindrical, straight or curved worms, coils, irregular flat and filled forms and the like.

15. The composition of pharmaceutical forms and excipients with sustained release and increase in drug bioavailability for poultry and pigs according to claim 14, characterized in that its form and appearance allows better acceptance by a bird or a pig.

16. A procedure for preparation of the composition of pharmaceutical forms and excipients with sustained release and increase in drug bioavailability for poultry and pigs according to claim 1, characterized in that comprise the steps of:
a) Dry mixing of the drug with the bioavailability promoting agent(s);
b) Adding one or more drug sustained-release polymers;
c) Mixing until mixture homogenization, adding colorants and flavorants when applicable;
d) Adding from 10 to 60% by weight of water total mixture;
e) Mixing until obtaining a mass of dry to semi-dry and soft consistency;
f) Extruding the mass in an extrusion equipment; and
g) Drying extruded products at room temperature, protected from light and air.

17. The procedure for preparation of a composition of pharmaceutical forms and excipients with sustained release and increase in drug bioavailability for poultry and pigs according to claim 16, further characterized in that in the stage of mass extrusion a nozzle is adapted to the extrusion machine with the physical form of the selected pharmaceutical form.
